(19) United States
(12) Patent Application Publication
(10) Pub. No.: US 2014/0360499 A1
(43) Pub. Date: Dec. 11, 2014

(54) Intranasal Airway Device

(71) Applicant: Ronal Peets, Sun City Center, FL (US)

(72) Inventor: Ronal Peets, Sun City Center, FL (US)

(21) Appl. No.: 14/299,209

(22) Filed: Jun. 9, 2014

Related U.S. Application Data
(60) Provisional application No. 61/832,858, filed on Jun. 9, 2013.

(51) Int. Cl. A61M 16/06; A61M 16/10

(52) U.S. Cl. CPC A61M 16/0672; A61M 16/10
USPC 128/204.12; 128/207.18

(57) ABSTRACT

An intranasal device having a first cannula for insertion into a first nostril of a user and a second cannula for insertion into a second nostril of a user. Each cannula has an interior surface defining a generally cylindrical lumen. On the exterior surface of each cannula is an anatomically conforming protrusion for engaging an anteromedial concavity of a nostril of the user and another anatomically conforming protrusion for engaging a posterolateral concavity of the nostril of the user.

### CLAIM OF PRIORITY

This application claims priority from U.S. Provisional Patent Application No. 61/832,858, entitled “Intranasal Airway Device”, filed on Jun. 9, 2013.

### FIELD

Embodiments of the invention relate generally to the field of nasal obstructions, and more specifically to a nasal airway device for reducing impediments to nasal airflow thereby reducing negative inhalation pressure in the body's airway.

### BACKGROUND

Since human noses have a limited range of sizes and anatomical detail, typical intranasal airway (INA) devices are similar in size and conformity. Such devices may be used to increase nasal airflow, for filtering air, delivering medication, and for swimming Typical INA devices include a pair of cannulae having a cylindrical or frustoconical shape and may include filters, valves, hoses, etc.

Conventional INA devices have many disadvantages. One disadvantage is the insertion and removal of the INA device. For example, a cylindrical INA device may be more difficult to insert and more difficult to insert farther into the nasal passage, and, while a frustoconical INA device may be inserted easier and farther, it is at the expense of decreased airflow. Also, conventional INA devices are typically removed by pulling on a connecting portion that connects the cannulae. This is often uncomfortable as it drags the cannulae against the septum.

Another disadvantage is that INA devices, whether cylindrically or frustoconically shaped, are difficult to retain in place due to nasal mucous and the pressure of exhalation. Additionally, INA devices are typically manufactured so as to have some mechanism for maintaining their shape during use. This may include manufacture from a rigid or semi-rigid material or a rigid support inserted or integrated with a pliable material. Such mechanisms, while helping to maintain the shape of the INA device and helping maintain increased airflow, also increase the propensity of the INA device to become unintentionally dislodged from the nasal passage.

Some conventional INA devices address the problem of device retention through use of an external retention device such as a strap or clip that holds the INA device to the user's head or nose. Such retention devices (e.g., retention strap) are uncomfortable and interfere with the user's activities. Moreover, some external retention devices (e.g., retention clip) decrease airflow.

Another conventional method of addressing device retention is to form a protrusion on the outer surface of the cannulae of the device. For some conventional devices such protrusions are directed toward the septum such that the protrusions on each cannula form a retaining clamp on the septum. Although, this helps to retain the INA device, it decreases airflow. Airflow is decreased because the protrusion causes a narrowing of the nasal passage, blocks a portion of the nasal passage used for airflow, and directs the aperture of each cannula inward, inhibiting airflow through the device to the nasal passage.

For other conventional devices the retaining protrusions are directed away from the septum and toward the inner surface of the lateral portions of the nose. Again, while this protrusion scheme helps to retain the INA device, it decreases airflow by blocking a portion of the nasal passage used for airflow, and directing the aperture of each cannula outward toward the inner surface of the lateral portions of the nose which likewise inhibits airflow through the device to the nasal passage.

Another distinct disadvantage of conventional INA devises is that in attempting to promote increased airflow and promote device retention the device instead causes a narrowing of the nasal passage resulting in decreased airflow by compressing the cannula which narrows the lumen. Moreover, the inclusion of such retention protrusions requires a narrower lumen, thus resulting in reduced airflow.

A distinct and major disadvantage of all prior art devices is that they fail to address the problems that arise when the nostrils, for various reasons are inadequate for the prevailing required airflow. Under such circumstances, a progressive accumulation of excessive negative inhalation pressure may occur. This may cause narrowing of the body's compliant airway. The increased negative pressure narrows the compliant airway and reduces nasal airflow. The restricted flow of ambient air may cause the user to inhale more forcefully and enter a cycle of increased negative inhalation pressure with an increase of the partial vacuum pressure on the compliant portions of the nasal passage which may decrease the level of available oxygen.

### SUMMARY

An intranasal airway device having two cannulae connected by a connecting portion. Each cannula has an approximately cylindrically shaped lumen. Each lumen has a diameter, depending upon the size of the device, which ranges from approximately 7 mm to 15 mm. The outer surface of the each cannula has one or more anatomically conforming protrusions.

For one embodiment of the invention the design features reduce impediments to the flow of ambient air at ambient pressure. For one such embodiment, the design features reduce the likelihood of typical negative inspiratory pressure progressing to the point of constricting the body's airway. Such an embodiment, therefore, reduces the likelihood that the user will experience oxygen deprivation.

For one embodiment of the invention, the outer surface of the each cannula has two anatomically conforming protrusions that project toward the anatomical cul-de-sac concavities inside the nostrils to engage the anteromedial and posterolateral cul-de-sac concavities, which are consistent characteristics of the typical human nose. The anatomically conforming protrusions support the nostrils in a wide open posture and provide increased airflow relative to prior art designs, while providing device retention and positioning.

For one embodiment of the invention, each cannula has a first anatomically conforming protrusion and a second anatomically conforming protrusion. The first anatomically conforming protrusion is directed anteriorly to engage the anteromedial concave recess of the nostril. The second anatomically conforming protrusion is directed posteriorly and somewhat laterally away from the septum to engage the posterolateral recess of the nostril. The anatomically conforming protrusions project outwardly from the exterior of the cannulae in a plane that is approximately perpendicular to the centerline of the lumeni and the nasal septum and approximately parallel to the base of the device. For one such embodiment, each cannula further includes a relatively small protrusion directed toward the nasal septum.

Embodiments of the invention help to lessen the buildup of negative inspiratory pressure in the body's air passageway to the lungs. The buildup of negative inspiratory pressure narrows that compliant passageway, further increasing inspiratory effort in a crescendo cycle which may cause hypoxia.

### BRIEF DESCRIPTION OF THE DRAWINGS

The foregoing summary, as well as the following detailed description of preferred embodiments, is better understood when read in conjunction with the appended drawings. For the purpose of illustrating the invention, there is shown in the drawings exemplary constructions of the invention; however, the invention is not limited to the specific methods and instrumentalities disclosed. In the figures of the accompanying drawings like reference numerals refer to similar elements. In the drawings:

FIG. 1 illustrates an intranasal airway device in accordance with one embodiment of the invention;

FIG. 2 illustrates an INA device in accordance with one embodiment of the invention;

FIG. 3 illustrates a cutaway side view of a cannula of an INA device in accordance with one embodiment of the invention;

FIG. 4 illustrates a perspective view of a cannula 400 of an INA device in accordance with one embodiment of the invention; and

FIG. 5 illustrates a cutaway lateral view of a cannula, in-place, in a right nostril of a user's nose in accordance with one embodiment of the invention.

### DETAILED DESCRIPTION

In the following description, numerous specific details are set forth. However, it is understood that embodiments of the invention may be practiced without these specific details. In other instances, well-known circuits, structures and techniques have not been shown in detail in order not to obscure the understanding of this description.

Reference throughout the specification to “one embodiment” or “an embodiment” means that a particular feature, structure, or characteristic described in connection with the embodiment is included in at least one embodiment of the present invention. Thus, the appearance of the phrases “in one embodiment” or “in an embodiment” in various places throughout the specification are not necessarily all referring to the same embodiment. Furthermore, the particular features, structures, or characteristics may be combined in any suitable manner in one or more embodiments.

Moreover, inventive aspects lie in less than all features of a single disclosed embodiment. Thus, the claims following the Detailed Description are hereby expressly incorporated into this Detailed Description, with each claim standing on its own as a separate embodiment of this invention.

Embodiments of the invention are applicable in a variety of settings in which increased or improved nasal airflow is desired.

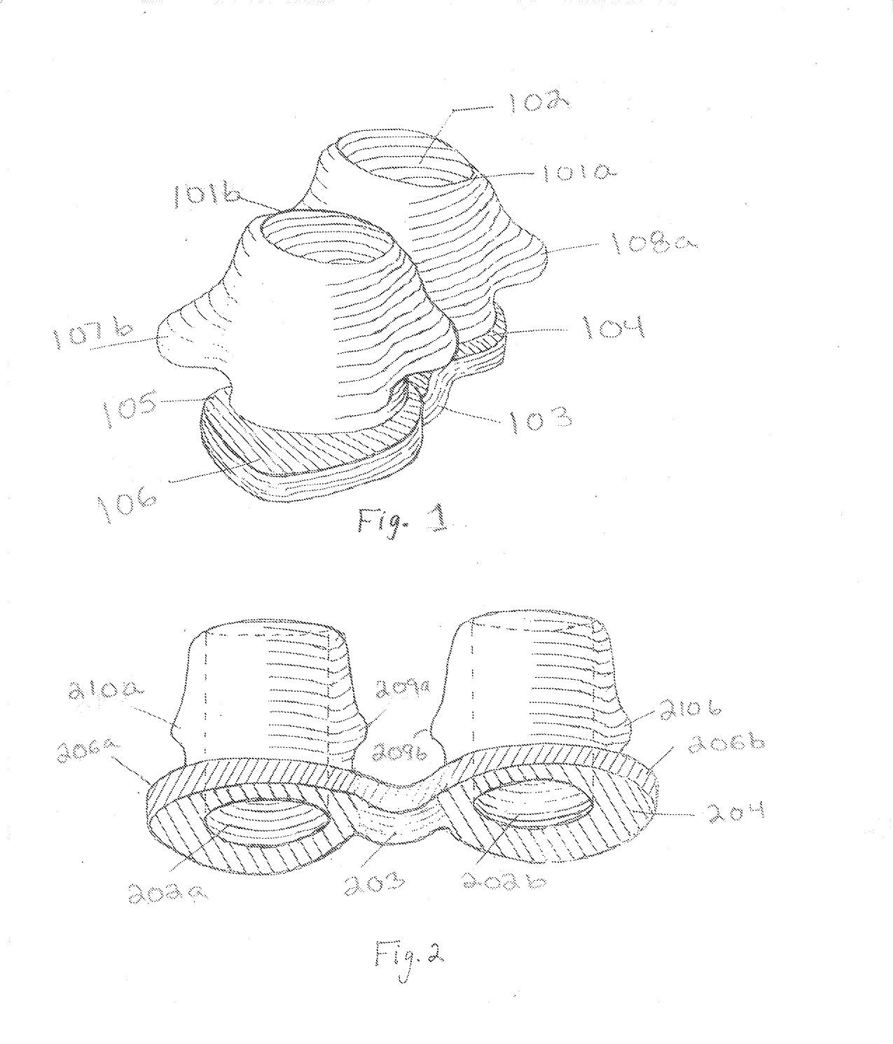

FIG. 1, illustrates an intranasal airflow (INA) device in accordance with one embodiment of the invention. As shown in FIG. 1, the INA device includes cannula 101a and cannula 101b that are open at both an insertion end 102 and an exterior end (not shown). The lumen of the cannulae 101a and 101b have an approximately uniform diameter throughout their length. The interior of the cannulae is substantially cylindrically shaped. For various embodiments the lumeni range in size from approximately 7 mm to 14 mm. The cannulae range in length from approximately 1.2 cm to 2.6 cm for various embodiments. The cannulae are connected to each other by a connecting portion (bridge) 103. The connecting portion separates the cannula a distance of approximately 4 mm to 12 mm depending on the size of the device for various embodiments. For one embodiment, each cannula has a base, shown, for example as base 104. The base supports the cannula and may include anterior flanges 105 and lateral flanges 106. For one embodiment, the interior flanges 105 and lateral flanges 106 may be used to hold and manipulate the INA device during insertion or removal, or to adjust the INA device during use. Each of the cannula may include anatomically conforming protrusions, shown, for example, as anatomically conforming protrusions 107b and 108a.

As shown in FIG. 1, the anatomically conforming protrusion 107b is directed anteriorly to engage the anteromedial concave recess of the nostril (not shown) and the anatomically conforming protrusion 108a is directed posteriorly and laterally to engage the posterolateral recess of the nostril. The anatomically conforming protrusions project outwardly from the exterior of the cannulae in a plane that is approximately perpendicular to the centerline of the lumeni and the nasal septum and approximately parallel to the base of the device.

For one embodiment of the invention the anatomically conforming protrusions 107b and 108a help to reduce impediments to the flow of ambient air at ambient pressure. For one such embodiment, anatomically conforming protrusions 107b and 108a reduce the likelihood of typical negative inspiratory pressure progressing to the point of constricting the body's airway. The anatomically conforming protrusions 107b and 108a also help to retain the INA device in-place during use.

FIG. 2 illustrates an INA device in accordance with one embodiment of the invention. As shown in FIG. 2, the connecting portion 203 may be curved to accommodate the columella of the user's nose. Also shown in FIG. 2 are additional protrusions 209a and 209b, and 210a and 210b, which engage the user's septum when the INA device has been inserted. The additional protrusions help to retain the device in-place during use. As shown in FIG. 2, septum engaging protrusions 209a and 209b, and 210a and 210b, are relatively small compared to anatomically conforming protrusions 107b and 108a, discussed above in reference to FIG. 1. For various embodiments, no additional protrusions are included and the INA device is adequately retained with the anatomically conforming protrusions 107b and 108a helping to retain the INA device in-place during use, as discussed above. FIG. 2 also illustrates exterior ends 202a and 202b of the respective cannulae of the INA device as well as base 204, and lateral flanges 206a and 206b.

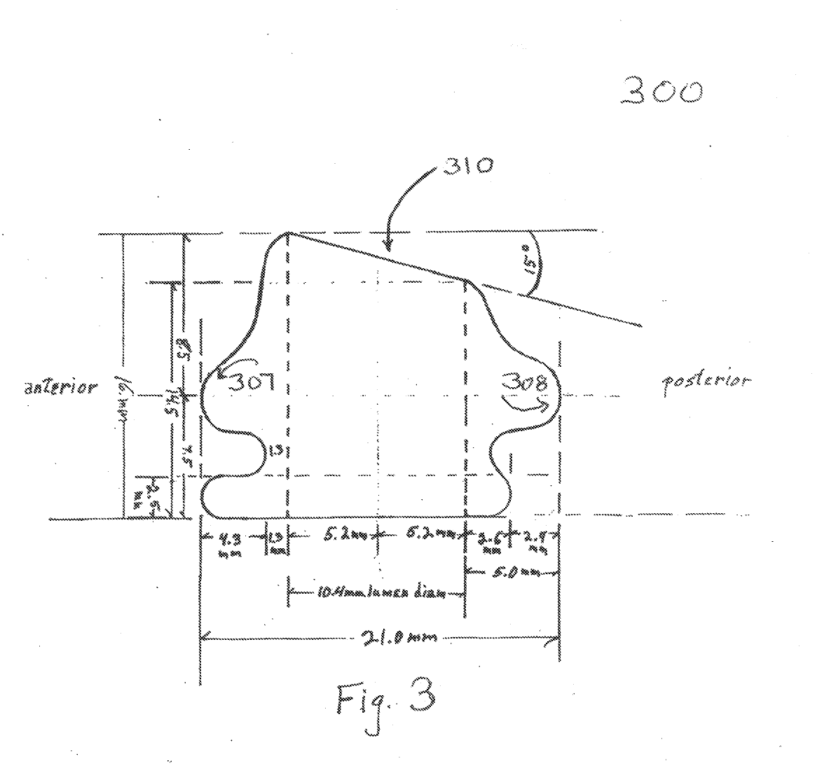

FIG. 3 illustrates a cutaway side view of a cannula 300 of an INA device in accordance with one embodiment of the invention. As shown in FIG. 3, the lumen 310 of the cannula 300 has a diameter of 10.4 mm. For one embodiment the cannula may be longer at the anterior end than at the posterior. For example, as shown in FIG. 3, the cannula is approximately 16 mm in length at the anterior end and decreases in length to approximately 14.5 mm at the posterior end, with the top end (insertion end) of the cannula sloping at approximately 15 degrees from the anterior end to the posterior end. As shown in FIG. 3, cannula 300 includes an anterior anatomically conforming protrusion 307 (anterior protrusion 307) that protrudes approximately 5.6 mm from the lumen 310 of cannula 300. The anterior protrusion 307 is positioned on the exterior surface cannula 300 approximately 7.5 mm from the base (i.e. the edge of the base) of cannula 300.

As shown in FIG. 3, cannula 300 also includes a posterior anatomically conforming protrusion 308 (posterior protrusion 308) that protrudes approximately 5 mm from the lumen 310 of cannula 300. The posterior protrusion 308 is also positioned on the exterior surface of cannula 300 approximately 7.5 mm from the base of cannula 300. For various alternative embodiments, the anterior protrusion 307 and the posterior protrusion 308 may be positioned at various distances from the base of the cannula and the distance from the base of the cannula 300 may not be the same for anterior protrusion 307 as for posterior protrusion 308.

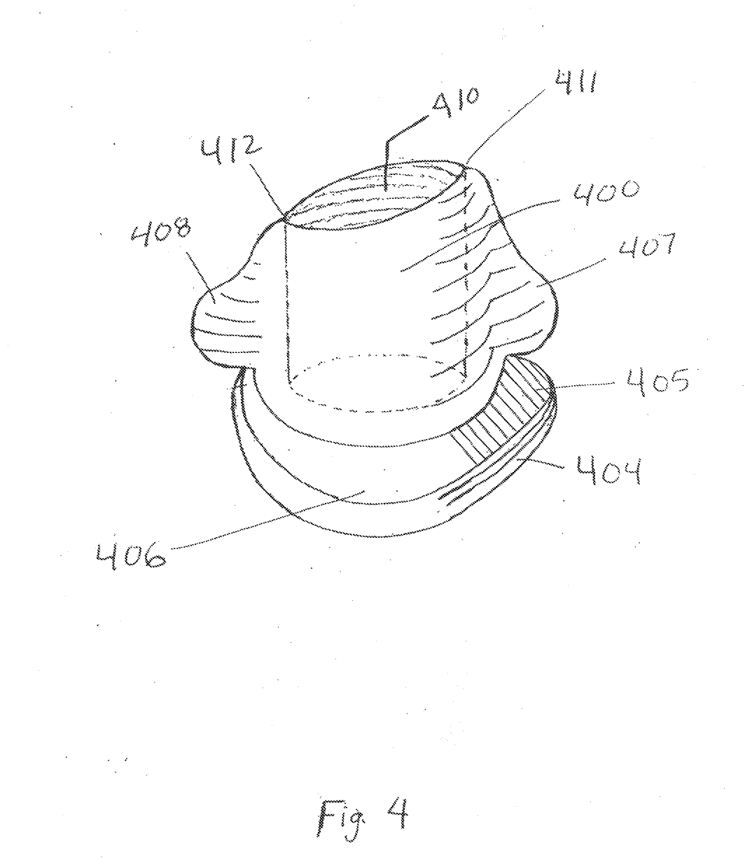

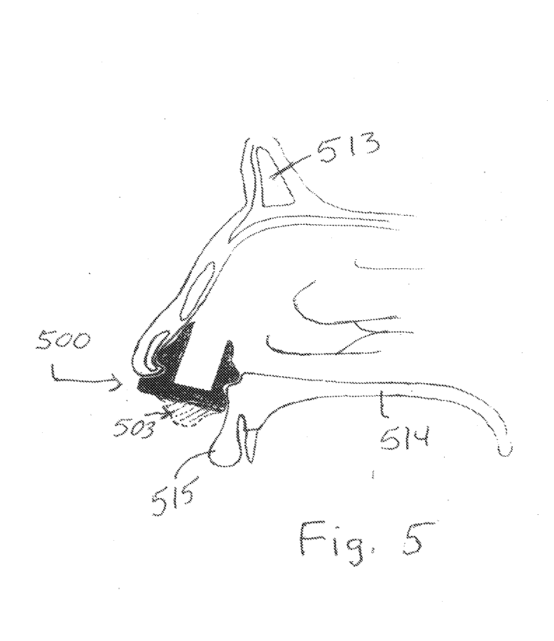

FIG. 4 illustrates a perspective view of a cannula 400 of an INA device in accordance with one embodiment of the invention. As shown in FIG. 4, cannula 400 illustrates a cannula for the right nostril of the user. Cannula 400 has an anterior end 411 that is longer than a posterior end 412, with the cannula 400 sloping from anterior end 411 to posterior end 412, as discussed above in reference to FIG. 3. Cannula 400 also includes a base portion 404 having an anterior flange 405 and a lateral flange 406.

Cannula 400 also includes an anterior protrusion 407 and a posterior protrusion 408. Anterior protrusion 407 and a posterior protrusion 408 are anatomically conforming protrusions that engage, respectively, the anteromedial and posterolateral cul-de-sac concavities of the typical human nose. Anterior protrusion 407 and a posterior protrusion 408 help support the nostrils in an open posture and provide increased airflow.

FIG. 5 illustrates a cutaway lateral view of a cannula 500 in-place in a right nostril of a user's nose in accordance with one embodiment of the invention. As shown in FIG. 5, cannula 500 includes a connecting portion 503. The connecting portion 503 obscures the columella of the user's nose. FIG. 5 illustrates the user's sinus 513, palate 514, and upper lip 515 to reference orientation of the cannula 500 in-place during use. As shown in FIG. 5, the anterior protrusion and the posterior protrusion (not referenced) engage, respectively, the anteromedial and posterolateral cul-de-sac concavities of the typical human nose (not referenced), to help support the nostrils in an open posture and provide increased airflow.

### General Matters

Embodiments of the invention provide an INA device for use in a variety of settings in which increased or improved nasal airflow is desired.

Although embodiments of the invention have been generally described above, various alternative embodiments are possible. For example, although an embodiment of the invention is described above as including an anterior protrusion and a posterior protrusion, alternative embodiments may have only one or the other of such protrusions. Further, although an embodiment of the invention is described above as including a base portion having anterior and lateral flanges, alternative embodiments may have only one or the other of such flanges or no flanges. Additionally, embodiments may include only one cannula, for use in one nostril. For such an embodiment the connecting portion or bridge may not be required. In alternative embodiments, a cannula in accordance with an embodiment of the invention may be used in conjunction with nasal delivery systems for medication or oxygen. For example, such nasal delivery systems typically include a nosepiece for insertion into a nostril of a user with a substance supply unit connected to the nosepiece, and a delivery mechanism for delivering a substance contained in the substance supply unit to the nostril of the user via the nosepiece. For one embodiment of the invention, the nosepiece of a system includes a cannula having an interior surface defining a generally cylindrical lumen and an exterior surface having one or more anatomically conforming protrusions as discussed above in reference to alternative embodiments of the invention. For such embodiments the cannula in accordance with such an embodiment may be employed to increase nasal airflow to improve delivery of the medication, oxygen, or other substance delivered intra-nasally.

For one embodiment the INA device may be constructed of a single material such as, for example, a biologically inert isomer of silicone, urethane, or Krayton. For alternative embodiments, the INA device may be constructed from one or more other suitable materials and may be constructed from multiple components.

While the invention has been described in terms of several embodiments, those skilled in the art will recognize that the invention is not limited to the embodiments described, but can be practiced with modification and alteration within the spirit and scope of the appended claims. The description is thus to be regarded as illustrative instead of limiting.

What is claimed is:

1. An intranasal device comprising:
a first cannula for insertion into a first nostril of a user;
a second cannula for insertion into a second nostril of a user;
each of the first cannula and the second cannula having an interior surface defining a generally cylindrical lumen and an exterior surface having a first anatomically conforming protrusion that engages an anteromedial concavity of a nostril of the user and a second anatomically conforming protrusion that engages a posterolateral concavity of the nostril of the user.

2. The device of claim 2 further comprising:
a second cannula for insertion into a first nostril of a user;
and a connecting portion, the connecting portion connecting the first cannula to the second cannula.

3. The intranasal device of claim 2 wherein each of the first cannula and the second cannula have a supporting base, each supporting base having an anterior flange and a lateral flange.

4. The intranasal device of claim 3 wherein the connecting portion

5. The intranasal device of claim 3 wherein each of the exterior surface of the first cannula and the exterior surface of the second cannula have a septum-engaging protrusion.

6. The intranasal device of claim 3 wherein an insertion end each of the first cannula and the second cannula slopes at approximately 15 degrees from an anterior end to a posterior end.

7. A nasal delivery system for delivering a substance to a nasal cavity of a subject, comprising:
a nosepiece for insertion into a nostril of a user, the nosepiece including a cannula having an interior surface defining a generally cylindrical lumen and an exterior surface including an anatomically conforming protrusion for engaging an anteromedial concave recess of a nostril;
a substance supply unit connected to the nosepiece; and
a delivery mechanism for delivering a substance contained in the substance supply unit to the nostril of the user via the nosepiece.

8. The nasal delivery system of claim 7 wherein the exterior surface of the cannula further includes a second anatomically conforming protrusion for engaging a posterolateral concavity of a nostril.

9. The nasal delivery system of claim 8 wherein the exterior surface of the cannula further includes a septum-engaging protrusion.

10. The nasal delivery system of claim 9 wherein an insertion end the cannula and slopes at approximately 15 degrees from an anterior end to a posterior end.

11. An intranasal device comprising:
a first cannula for insertion into a first nostril of a user;
a second cannula for insertion into a second nostril of a user, each of the first cannula and the second cannula having an interior surface defining a generally cylindrical lumen and an exterior surface having a first anatomically conforming protrusion that engages an anteromedial concavity of a nostril of the user and a second anatomically conforming protrusion that engages a posterolateral concavity of the nostril of the user.

12. The intranasal device of claim 11 wherein each of the first cannula and the second cannula have a supporting base, each supporting base having an anterior flange and a lateral flange.

13. The intranasal device of claim 12 wherein the connecting portion

14. The intranasal device of claim 12 wherein each of the exterior surface of the first cannula and the exterior surface of the second cannula have a septum-engaging protrusion.

15. The intranasal device of claim 12 wherein an insertion end each of the first cannula and the second cannula slopes at approximately 15 degrees from an anterior end to a posterior end.
